(19) United States
(12) Patent Application Publication
(10) Pub. No.: US 2025/0186037 A1
(43) Pub. Date: Jun. 12, 2025

(54) BONE ANCHOR LINKING METHODS AND MATERIALS

(71) Applicant: SPEED CLIP SOLUTIONS, LLC, Dallas, TX (US)

(72) Inventors: Kevin KRUSE, Dallas, TX (US); Lawrence BINDER, Miami, FL (US)

(73) Assignee: SPEED CLIP SOLUTIONS, LLC, Dallas, TX (US)

(21) Appl. No.: 19/061,688

(22) Filed: Feb. 24, 2025

Related U.S. Application Data
(63) Continuation of application No. PCT/US2023/072692, filed on Aug. 23, 2023.
(60) Provisional application No. 63/373,284, filed on Aug. 23, 2022.

(51) Int. Cl. A61B 17/04; A61B 17/06; A61F 2/08

(52) U.S. Cl. CPC A61B 17/0401; A61B 17/06166; A61F 2/0811; A61B 2017/0414; A61B 2017/0448; A61B 2017/0459; A61B 2017/0464; A61F 2002/0823; A61F 2002/0852; A61F 2002/0882

(57) ABSTRACT

A bone anchor linking method and materials are disclosed. The method of using a bone anchor linking including inserting a first anchor into a first hole in the bone, wherein the first anchor comprises one or more sutures, forming a second anchor using the one or more sutures from the first anchor, inserting the second anchor into a second hole in the bone, and pulling at least one suture tail of the one or more sutures exiting the second anchor. The suture includes a body with a first end, a second end, and a length, wherein at least a portion of the body between the first end and the second end has a larger cross sectional area than the cross sectional area at the first end and the second end. The anchor includes a first body and a second body inserted into the first body with a weave pattern.

### CROSS REFERENCE TO RELATED APPLICATION

This application is a continuation of PCT/US2023/072692 filed on Aug. 23, 2023 and entitled Bone Anchor Linking Methods and Materials, which claims priority benefit under 35 U.S.C. § 119(e) of U.S. provisional application No. 63/373,284 filed Aug. 23, 2022, which is incorporated herein by reference in its entirety.

### FIELD OF THE INVENTION

The present invention relates generally to general surgery, bone anchor linking systems used for soft tissue anchoring. More specifically, but not exclusively, the present invention relates to biased locking anchor mechanisms and systems, as well as the surgical methods for using the biased locking anchor mechanisms and systems.

### BACKGROUND OF THE INVENTION

Often when a patient's bone is injured there is also soft tissue damage. The currently available fixes for soft tissue damage are not always secure and can lead to the soft tissue not healing properly. Thus, improved anchoring devices and methods are needed to allow for soft tissue repair of soft tissue surrounding or associated with soft tissue injuries.

### SUMMARY OF THE INVENTION

Aspects of the present invention provide bone anchor linking systems, devices, implants, and methods for using the bone anchor linking systems, devices, and implants for anchoring soft tissue.

In one aspect, provided herein is a bone anchor linking method including inserting a first anchor into a first hole in the bone, wherein the first anchor comprises one or more sutures, forming a second anchor using the one or more sutures from the first anchor, inserting the second anchor into a second hole in the bone, and pulling at least one suture tail of the one or more sutures exiting the second anchor.

In another aspect, provided herein is a suture includes a body with a first end, a second end, and a length, wherein at least a portion of the body between the first end and the second end has a larger cross sectional area than the cross sectional area at the first end and the second end.

In yet another aspect, provided herein is an anchor includes a first body and a second body inserted into the first body with a weave pattern, wherein the weave pattern includes at least one first opening positioned along the length of the first body, at least one second opening positioned along the length of the first body and spaced apart in at least one direction from the at least one first opening, and wherein the second body is inserted into the at least one first opening, passes over an exterior surface of the first body, and is inserted into the at least one second opening.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1A shows top views of weave patterns for a biased locking anchor mechanism, in accordance with an aspect of the present disclosure;

FIG. 1B shows a top view of a weave pattern, in accordance with an aspect of the present disclosure;

FIG. 2 illustrates an anchor assembly placed in bone, in accordance with an aspect of the present disclosure;

FIG. 3 illustrates the anchor assembly of FIG. 2 with a sleeve for receiving suture tails from the anchor assembly, in accordance with an aspect of the present disclosure;

FIG. 4 illustrates the anchor assembly of FIG. 3 after the sleeve is placed into bone, in accordance with an aspect of the present disclosure;

FIG. 5 illustrates the anchor assembly of FIG. 2 with a fabric anchor placed into bone, in accordance with an aspect of the present disclosure;

FIG. 6 illustrates a suture with varying cross-sectional areas along its length, in accordance with an aspect of the present disclosure;

FIG. 7 illustrates a suture having a ridged surface along its length, in accordance with an aspect of the present disclosure;

FIG. 8A illustrates the suture of FIG. 6 inserted into an anchor placed into bone in a first position, in accordance with an aspect of the present disclosure;

FIG. 8B illustrates the suture, anchor and bone of FIG. 8A in a second position, in accordance with an aspect of the present disclosure;

FIG. 9 is a perspective view of a weave pattern for a biased locking anchor mechanism, in accordance with an aspect of the present disclosure;

FIG. 10 illustrates a bottom perspective view of a simulated bone portion with two anchors, in accordance with an aspect of the present disclosure;

FIG. 11 illustrates a top perspective view of the simulated bone portion of FIG. 10 including the suture threads in a first position, in accordance with an aspect of the present disclosure;

FIG. 12 illustrates a top perspective view of the simulated bone portion of FIG. 10 with the suture threads in a second position, in accordance with an aspect of the present disclosure;

FIG. 13 illustrates a top perspective view of the simulated bone portion of FIG. 10 with the suture threads in a third position, in accordance with an aspect of the present disclosure;

FIG. 14 illustrates a top perspective view of the simulated bone portion of FIG. 10 with the suture threads in a fourth position, in accordance with an aspect of the present disclosure; and

FIG. 15 illustrates a top perspective view of the simulated bone portion of FIG. 10 with the suture threads in a fifth position, in accordance with an aspect of the present disclosure.

### DETAILED DESCRIPTION FOR CARRYING OUT THE INVENTION

Generally stated, disclosed herein are bone anchor linking methods. Further, biased locking anchor mechanisms, devices, and implants for use in the bone anchor linking methods are discussed.

In this detailed description and the following claims, the words proximal, distal, anterior, posterior, medial, lateral, superior and inferior are defined by their standard usage for indicating a particular part of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

Similarly, positions or directions may be used herein with reference to anatomical structures or surfaces. For example, the devices and methods, and the aspects, components, features and the like thereof, disclosed herein may be described with respect to one side of the body for brevity purposes. However, as the human body is relatively symmetrical or mirrored about a line of symmetry (midline), it is hereby expressly contemplated that the device and methods, and the aspects, components, features and the like thereof, described and/or illustrated herein may be changed, varied, modified, reconfigured or otherwise altered for use or association with another side of the body for a same or similar purpose without departing from the spirit and scope of the invention.

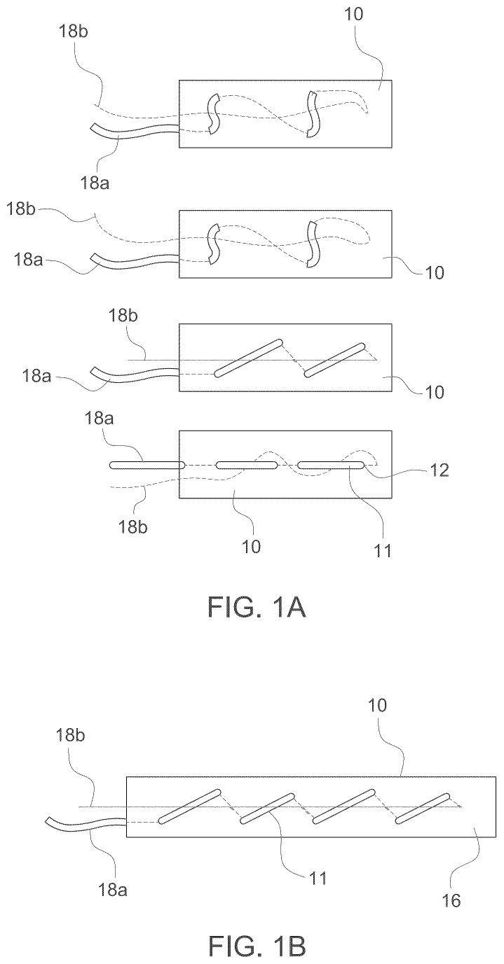

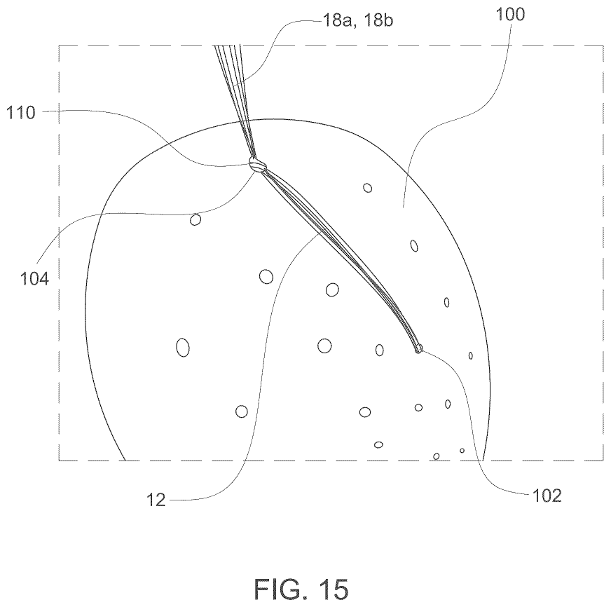

Referring to the drawings, wherein like reference numerals are used to indicate like or analogous components throughout the several views, and with particular reference to FIGS. 1-15, there are illustrated biased locking anchor mechanisms, devices, and implants for use in the bone anchor linking methods.

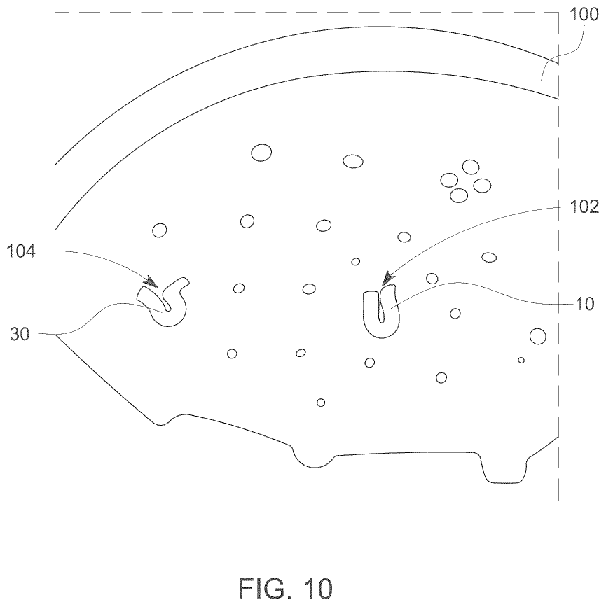

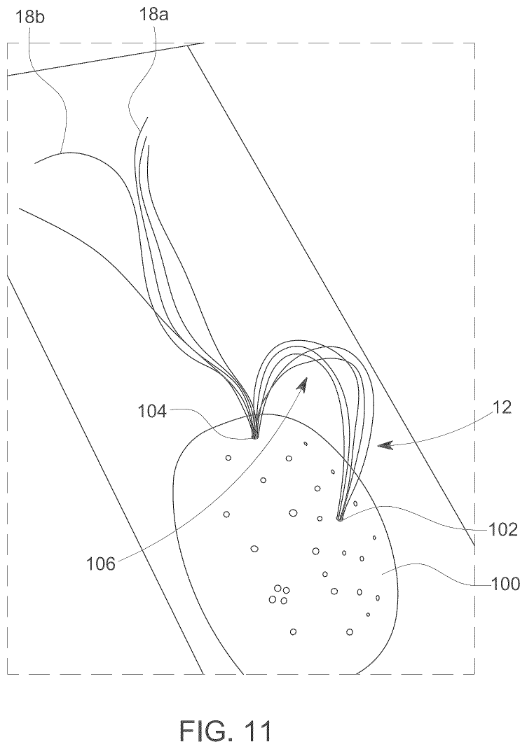

Referring now to FIGS. 1A-15, bone anchor linking methods are shown. Specifically, as shown in FIGS. 10-15, the method includes placing a first anchor 10 into a first hole 102 in a bone 100 of a patient. The first anchor 10 may then be set by pulling on the exiting suture tails 18a, 18b. Next, one or more of the exiting suture tails 18a, 18b from the first anchor 10 may be taken and pulled through a sleeve or fabric portion 15, 16. The sleeve 15, 16 may create a second anchor 30 during the surgical procedure. Then, the newly assembled second anchor 30 may be placed onto an insertion instrument (not shown). The second anchor 30 may be, for example, the same or similar to the first anchor 10, as described in greater detail below. The second anchor 30 may then be placed into a second hole 104 in the patient's bone 100, as shown in FIG. 10. Once the second anchor 30 is placed, one or more sutures 18a, 18b will continuously span between the first anchor 10 and the second anchor 30, as shown in FIGS. 11-15. If additional anchors 10, 30 are needed, then one or more of the exiting suture tails 18a, 18b from the second anchor 30 or any subsequent anchor may be taken and pulled through a sleeve 15, 16 creating an additional anchor 10, 30. The additional anchors 10, 30 may then be placed onto the insertion instrument (not shown) and inserted into an additional hole in the patient's bone 100. The span between each anchor 10, 30 can be used to hold down soft tissue or for other surgical purposes as understood by one of ordinary skill in the art. Once the desired number of anchors 10, 30 are placed into the patient's bone 100, then the suture tails 18a, 18b existing the final anchor can be locked with a clip (not shown) or tied to prevent loosening of the construct. The clip may be, for example, as described in U.S. Provisional Patent Application No. 63/253,869 filed Oct. 8, 2021 or U.S. Pat. No. 11,311,284 issued Apr. 26, 2022, which are both incorporated herein in their entireties.

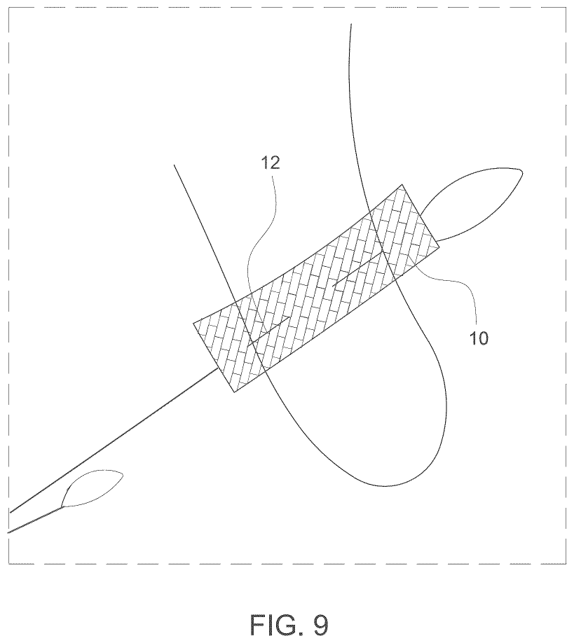

Referring now to FIGS. 1A-1B, additional details regarding the anchors 10, 30 being used to make or form biased locking anchor mechanisms are shown. For example, FIGS. 1A-1B shows various weave patterns of a suture 12 that may be used to make a biased locking anchor mechanism from a fabric anchor 10. The suture 12 includes loops 11 and suture limbs or suture tails 18a, 18b at each end of the suture 12. As illustrated, the dashed lines of suture 12 are positioned on the far side or bottom of the anchor 10 and the thick lines of suture 12 are positioned on the top of the anchor 10. FIG. 1A shows that the holes in a patient's bone can be in line or at angles from each other. In addition, FIG. 9 shows the holes in the patient's bone in line. Whether the holes are in line or at angles, the main line or second end of the suture tail 18b should be positioned under the loops 11. By positioning the suture tail 18b under the loops 11, the biased locking anchor mechanism will provide a biased amount of friction when the suture tail or first end 18a is pulled versus the second end 18b. The biased locking anchor mechanism may have any number of anchors 10, 30 to provide the desired number of loops 11 for securement of soft tissue of the patient. As shown in FIG. 1A, the biased locking anchor mechanisms each include two loops 11, while FIG. 1B shows a biased locking anchor mechanism with four loops 11. In addition, the number of holding loops 11 can be increased as needed to increase the friction required to prevent slippage of the suture, or sutures woven into the anchor 10.

The first body, sleeve 15, or fabric portion 16 may be made of a woven or braided fabric material of densely packed fibers of medical grade polymers. The medical grade polymers may be, for example, UHMWPE, polyester, PTFE, PEEK, nylon, or another medical grade woven polymer. The first body 15, 16 may have a generally rectangular outer profile, as shown in FIGS. 1A, 1B and 9. The ends of the first body 15, 16 may be, for example, tapered, rounded or chamfered. At least one end of the first body 15, 16 may be, for example, a thickened region to allow for grasping and insertion. The first body 15, 16 may have a series of at least two or more holes. The holes may be, for example, reinforced holes. The holes may be manufactured along the length of the first body 15, 16 for passing a suture or suture material 12 through the first body 15, 16. The holes may be positioned, for example, in line, at an angle, or generally arranged as shown in FIGS. 1A-1B. The first body 15, 16 may also include an insertion hole extending through the length of the first body 15, 16. The insertion hole may be manufactured at one end for loading on an insertion instrument for placement into a hole 102, 104 in a bone 100. The first body 15, 16 may be, for example, approximately 0.2 mm to 5 mm thick, more preferably approximately 1 mm thick. The outer rectangular profile of the first body 15, 16 may be, for example, approximately 1 mm to 20 mm wide and approximately 5 mm to 60 mm long. In one embodiment the outer rectangular profile of the first body 15, 16 may be, for example, approximately 5 mm wide and approximately 25 mm long.

In another embodiment, there is another body 15, 16 may have a mostly cylindrical cross section, which may have sutures 12 funneled through the central region. The sutures 12 may enter and leave the sleeve 15, 16 in various locations designed to deform the shape of the sleeve 15, 16 when the sutures 12 are tensioned. The other body 15, 16 has a length of, for example, approximately 4 mm to 80 mm, and preferably approximately 40 mm. The generally cylindrical diameter of the sleeve 15, 16 may be, for example, approximately 0.5 mm to 10 mm, and preferably approximately 2.5 mm. The cylindrical diameter will depend on the quantity and size of the mating assembly components.

The second elongated body or suture 12 may be, for example, one or more bodies or sutures made of a suture or suture like material. The second body 12 is passed through the first body 10, 30 in a specific pattern of entering and exiting the first body 10, 30. The specific pattern of the second body 12 causes the first body 10, 30 to fold onto itself. The specific pattern of the second body 12 also allows the second body 12 to slip when one suture limb 18a is pulled and locks or has a higher resistance to slipping when the opposite suture limb or limbs 18b is pulled. The ratio of slipping to not slipping may be, for example, approximately 1:2-1:20, more preferably approximately 1:2, 1:5, 1:10, 1:20. The ratio of slipping may be determined based on the weave pattern, material friction, texture, shape, and denier of the second body 12. The ratio of slipping will always be greater than 1:1 of the coefficient of friction and resistance to pulling force when comparing pulling one end of the sutures to the other. Thus, there will always be a bias to one of the ends 18a, 18b that are pulled. The second body 12 may have a perpendicular cross-sectional area that changes larger or smaller along the length of the suture limb 18a, 18b. More specifically, the suture 12 may have, for example, a smaller cross-sectional area at the ends 18a, 18b and may transition to a larger cross-sectional area towards the central region of the suture 12. In addition, the second body 12 may have a generally round, flat, rectangular, or square cross section. For a second body 12 with a round or square cross section, the cross section may be, for example, approximately 0.1 mm to 3 mm, more preferably approximately 0.5 mm to 1.5 mm. For a second body 12 with a rectangular cross section, the cross section may be, for example, approximately 0.05 mm to 5 mm, more preferably approximately 0.5 mm to 1.5 mm. The second body 12 may also transition from various cross-sectional shapes and sizes along the length of the body 12. In addition, the second body 12 has an overall longitudinal length of, for example, approximately 10″ to 100″ with a preferred length of approximately 50″.

The first body 15, 16 and second body 12 can be assembled to be used as a bone anchor or anchor assembly 10, 30 for securing soft tissue to bony anatomy during surgical procedures. The anchor assembly 10, 30 made from the first body 15, 16 and the second body 12 may use suture limbs 18a, 18b from a third body anchor assembly 10, 30 that is first placed in a bone 100. Next, the first and second body assemblies 15, 16, 12 may be put together by someone in the operating room during the surgical procedure. During an intraoperative assembly process suture shuttles or nitinol wires may be used to place the second body 12, or bodies, in the predefined locations within the first body 15, 16.

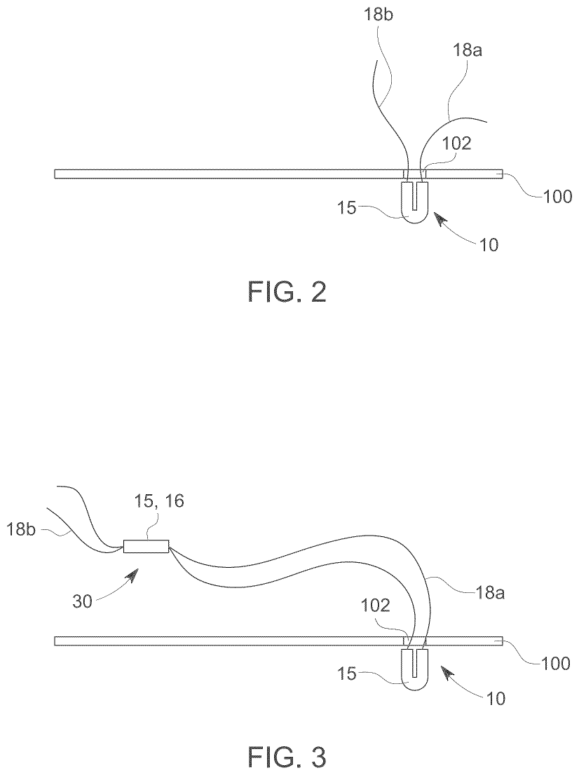

Referring now to FIG. 2, an anchor assembly 10 having a sleeve 15 with a suture 18a, 18b placed into bone 100 and two suture limbs 18a, 18b extending out from the anchor 10 superior to the bone 100 is shown. Although the sleeve 15 is shown in FIG. 2, it is also contemplated that the anchor assembly 10 may include a fabric portion 16, as shown in FIG. 1B. As illustrated, once the sleeve 15 is inserted into bone 100, the sleeve 15 curves to form a surface area larger than the opening 102 to secure the anchor 10 to the bone 100.

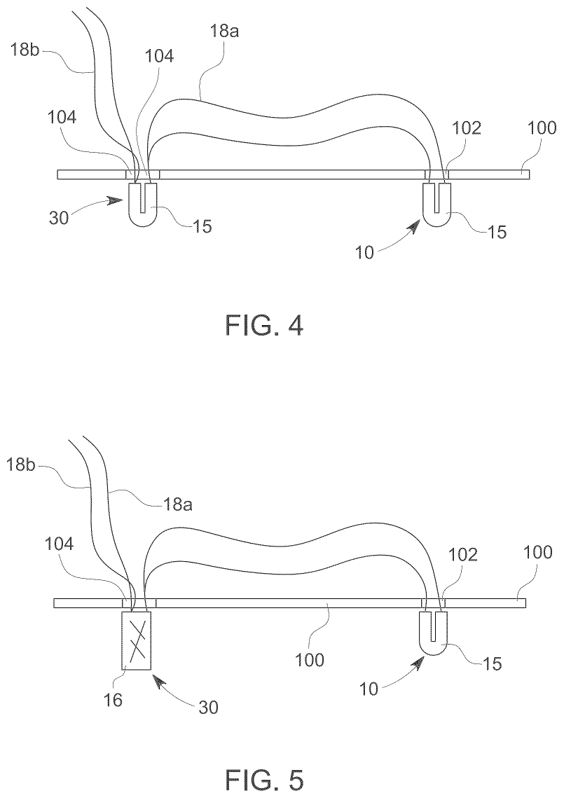

As shown in FIG. 3, the two suture limbs 18a, 18b can be placed through a second sleeve 15 or piece of fabric 16. FIG. 4 shows the tubular sleeve 15 placed into the bone 100 through opening 104. While FIG. 5 shows the fabric anchor 16 placed into the bone 100. After placement of an anchor 10 in the bone 100 the suture limbs 18a, 18b may be locked to prevent further slippage. The suture limbs 18a, 18b may be locked by (1) placing a clamping mechanism on the exposed suture ends 18a, 18b; (2) tying a knot using the suture ends 18a, 18b; (3) tying a knot using the suture ends 18a, 18b and the suture material that transitions between the two anchors 10, 30; or (4) pulling the suture limbs 18a, 18b back into the second anchor 30 using a shuttle suture.

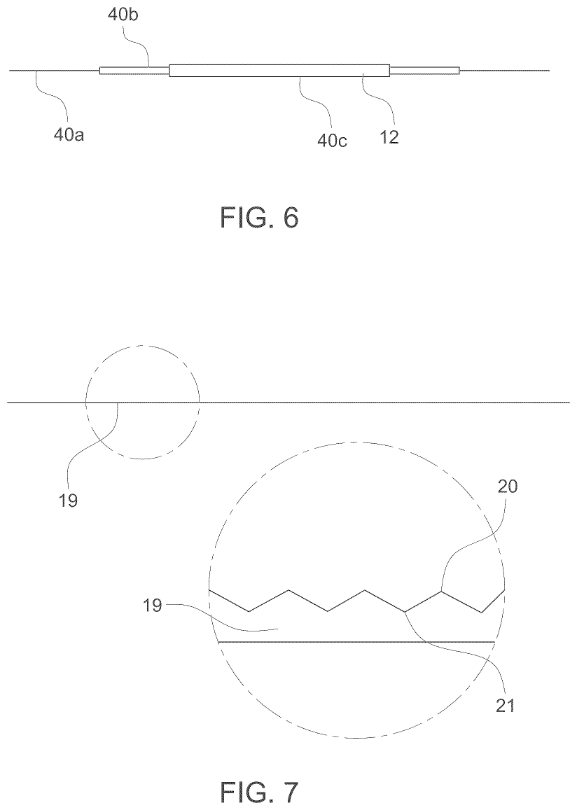

In some embodiments, the suture 12 may have a varying cross-sectional area along its length, such as shown in FIG. 6. The suture 12 may include two suture tails or limbs 40a, 40b at each end of the length and an intermediate suture portion 40c. The intermediate suture portion 40c may have, for example, a larger cross-sectional area than the suture limbs 40a, 40b. Further, the cross-sectional area of suture limb 40b may be, for example, larger than the cross-sectional area of suture limb 40a. The at least one second body 12 is placed through the first body 15, 16 with the smallest cross section placed through the first body 15, 16. Then, the assembly 10, 30 of the bodies 12, 15, 16 is placed into a hole created in a bone 100 and tensioned to cause bunching and actuation of the sleeve 15, 16. As the suture limbs 40a, 40b are pulled, the larger cross section 40c of the second body 12 is gradually fed through the first body 15, 16 adding more material under the cortex, expanding the size of the first body 15, 16, making it more resistant to pulling back through the hole it was placed through. The assembly of the first body 15, 16 and the second body 12 may be done either (1) in the operating room during the surgical procedure and linked to an anchor already placed in bone via the second body limbs 40a, 40b; (2) during the procedure and not linked; or (3) prior to the procedure in a manufacturing environment.

Another embodiment of a suture 12 is shown in FIG. 7. The suture 12 shown in FIG. 7 includes a ridged surface 19. The ridged surface 19 is defined with peaks 20 and valleys 21, alternating along the length of the suture 12. The rigid surface 19 provides increased surface texture to assist with securing the anchor 10 under the bone 100. The second body 12 has mostly perpendicular ridges 19, for example, 1-90 degrees from the longitudinal axis, as shown in FIG. 7. The ridges 19 may run along some or all of the length of the suture 12. The distance between each peak 20 or valley 21 may be, for example, approximately 0.1 mm to 5 mm. The delta between the peaks 20 and valleys 21 may be, for example, approximately 0.1 mm to 1 mm.

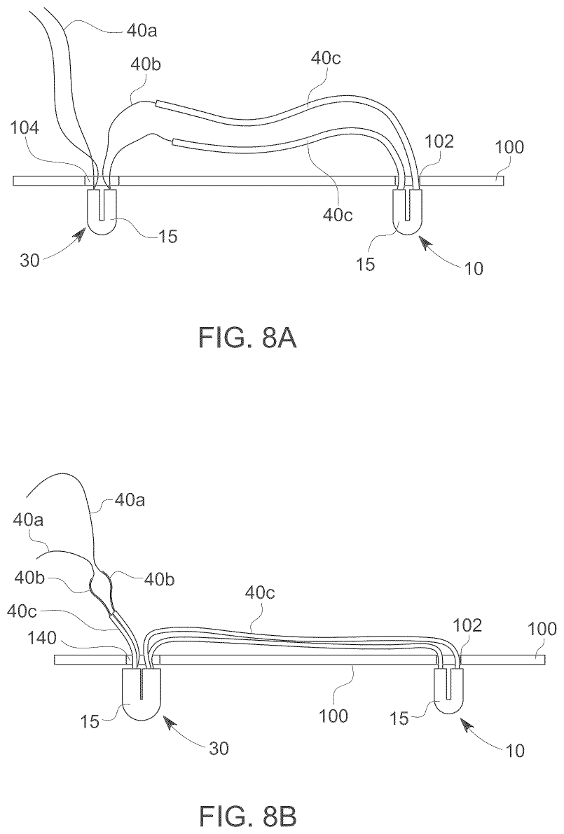

Referring now to FIGS. 8A-8B, the suture 12 of FIG. 6 is shown inserted into bone 100 using two anchors 10, 30. As shown in FIG. 8A, the suture 12 is threaded into the anchor 10 from the smaller cross-sectional area 40a. Once the suture 12 is coupled to the sleeve 15 or fabric portion 16 to form anchor 10, the anchor 10 is placed into the bone 100 through opening 102. Next, a suture limb 40a is pulled, introducing the sequentially larger cross-sectional regions (40a, 40b, 40c) into the anchor 10 as the anchor 10 is positioned below the surface of the bone 100. As the suture limb 40a is pulled, the amount of material positioned within the sleeve 15 increases, thereby increasing the size of the sleeve 15 beneath the surface of the bone 100. The suture limbs 40a may then be threaded through a second anchor 30 and the second anchor 30 placed within an opening 104 in the bone 100.

As shown in FIG. 8B, the suture limb 40a may be pulled to move the larger cross-sectional areas 40b, 40c of the suture 12 through and into the sleeve 15 until the cross-sectional area 40c is positioned within the sleeve 15. As the cross-sectional areas 40b, 40c are pulled through the sleeve 15, the size of the sleeve 15 beneath the surface of the bone 100 increases. The increased sleeve 15 size assists with securing the anchor 30 below the bone 100.

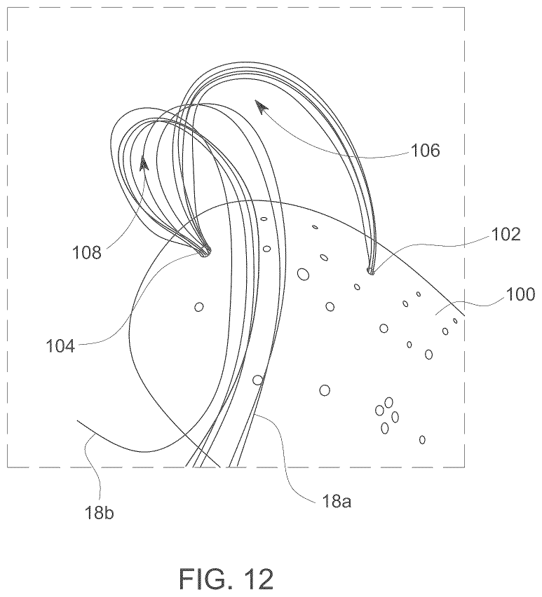

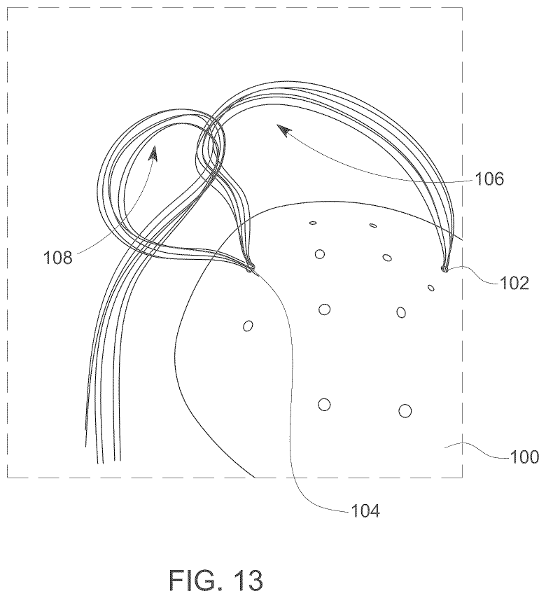

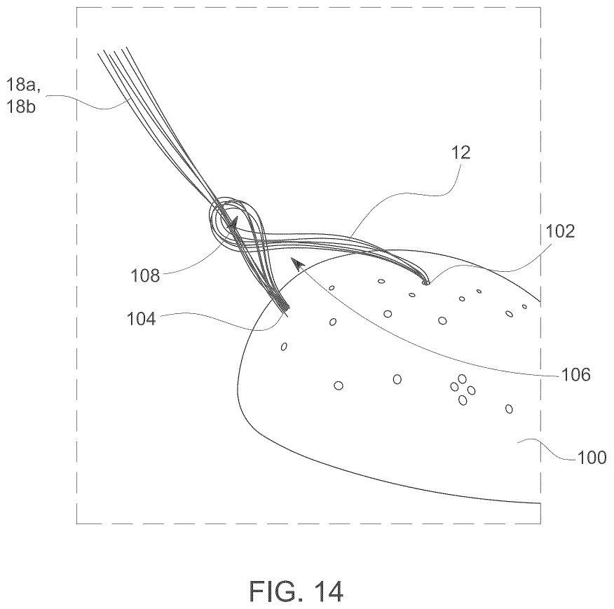

Referring now to FIGS. 10-15, the suture limbs 18a, 18b being tied in a knot after insertion of at least two anchors 10, 30 is shown. FIG. 11 shows the first anchor 10 inserted through opening 102 in the bone 100 and the second anchor 30 inserted through opening 104 in the bone 100. The plurality of sutures 12 extend from the opening 102 to the opening 104 forming a first loop 106 above the bone 100 and the suture limbs 18a, 18b extend away from the bone 100 from opening 104. As shown in FIG. 12, the suture limbs 18a, 18b may be inserted through the first loop 106 to form a second loop 108 between the second opening 104 and the suture limbs 18a, 18b. Referring now to FIG. 13, the suture limbs 18a, 18b are then inserted back through the second loop 108. Then, the suture limbs 18a, 18b are pulled to tighten the suture 12 across the bone 100, as shown in FIG. 14. Finally, pulling the suture limbs 18a, 18b is complete and the suture 12 is pulled tight across the top of bone 100 with a knot 110 securing the suture 12 in place above opening 104.

As may be recognized by those of ordinary skill in the art based on the teachings herein, numerous changes and modifications may be made to the above-described and other embodiments of the present disclosure without departing from the scope of the disclosure. The devices, implants, and/or systems as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the devices, implants and systems may include more or fewer components or features than the embodiments as described and illustrated herein. Accordingly, this detailed description of the currently-preferred embodiments is to be taken in an illustrative, as opposed to limiting of the disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. A method of holding soft tissue to bone, comprising:
inserting a first anchor into a first hole in the bone, wherein the first anchor comprises one or more sutures;
forming a second anchor using the one or more sutures from the first anchor;
inserting the second anchor into a second hole in the bone; and
pulling at least one suture tail of the one or more sutures exiting the second anchor.

2. The method of claim 1, wherein inserting the first anchor into the first hole in the bone comprises:
placing the first anchor into the first hole in the bone; and
setting the first anchor by pulling the at least one suture tail.

3. The method of claim 1, wherein forming the second anchor comprises:
obtaining at least one of a sleeve and a fabric portion;
taking one or more of the at least one suture tail; and
inserting one or more of the at least one suture tail through the at least one of sleeve and fabric portion to form the second anchor.

4. The method of claim 1, wherein inserting the second anchor into the second hole in the bone comprises:
placing the second anchor into the second hole in the bone; and
setting the second anchor by pulling the at least one suture tail exiting the second anchor.

5. The method of claim 1, further comprising:
forming at least one third anchor; and
inserting the at least one third anchor into at least one third hole in the bone.

6. The method of claim 5, wherein the at least one suture tail exits a final anchor of the at least one third anchor.

7. The method of claim 6, further comprising:
locking the at least one suture tail leaving a final anchor.

8. The method of claim 7, wherein locking the at least one suture tail comprises:
inserting a clip over the at least one suture tail to secure the at least one suture tail in place.

9. The method of claim 7, wherein locking the at least one suture tail comprises:
tying the at least one suture tail to secure the at least one suture tail in place.

10. A suture, comprising:
a body with a first end, a second end, and a length, wherein at least a portion of the body between the first end and the second end has a larger cross sectional area than the cross sectional area at the first end and the second end.

11. The suture of claim 10, wherein the at least one portion of the body comprises:
a first portion positioned between the first end and the second end and having a first length and a first width, wherein a width at the first end and a width at the second end are smaller than the first width.

12. The suture of claim 11, wherein the at least one portion of the body further comprises:
a second portion positioned between the first portion and the first end and having a second length and a second width;
a third portion positioned between the first portion and the second end and having a third length and a third width;
wherein the first length is larger than the second length and third length;
wherein the first width is larger than the second width and third width; and
wherein the width at the first end and the width at the second end are smaller than the second width and the third width.

13. An anchor, comprising:
a first body; and
a second body, wherein the second body is inserted into the first body with a weave pattern;
wherein the weave pattern comprises:
at least one first opening positioned along the length of the first body; and
at least one second opening positioned along the length of the first body and spaced apart in at least one direction from the at least one first opening; and
wherein the second body is inserted into the at least one first opening, passes over an exterior surface of the first body, and is inserted into the at least one second opening.

14. The anchor of claim 13, wherein the first body is selected from a sleeve and a fabric portion.

15. The anchor of claim 13, wherein the second body is at least one suture.

16. The anchor of claim 13, wherein the at least one first opening is two first openings and the at least one second opening is two second openings, wherein each of the first openings and the second openings are positioned linearly along the length of the first body, and wherein the first openings are alternated with the second openings along the length of the first body.

17. The anchor of claim 13, wherein the at least one first opening is two first openings and the at least one second opening is two second openings, wherein each of the first openings and the second openings are positioned offset from each other along the length of the first body and the width of the first body, and wherein the first openings are alternated with the second openings along the length of the first body.

18. The anchor of claim 13, wherein the at least one first opening is two first openings and the at least one second opening is two second openings, wherein each of the first openings and the second openings are positioned offset from each other along the length of the first body and positioned linearly along the width of the first body, and wherein the first openings are alternated with the second openings along the length of the first body.

19. The anchor of claim 13, wherein the at least one first opening is at least two first openings and the at least one second opening is at least two second openings.

20. The anchor of claim 13, wherein the second body comprises:
an intermediate portion;
a first suture tail; and
a second suture tail;
wherein the first suture tail has a first resistance when pulled;
wherein the second suture tail has a second resistance when pulled; and
wherein the first resistance is larger than the second resistance.
